(19) United States
(12) Patent Application Publication
(10) Pub. No.: US 2015/0212054 A1
(43) Pub. Date: Jul. 30, 2015

(54) COMPACT MULTIPLE CHROMATOGRAPHIC MEDIA DEVICE

(71) Applicants: Paul SAUNDERS, (US); Alex SAUNDERS, (US)

(72) Inventors: Paul Alexander Saunders, Palo Alto, CA (US); Alex Michael Saunders, San Carlos, CA (US)

(21) Appl. No.: 14/419,424

(22) PCT Filed: Aug. 6, 2013
(86) PCT No.: PCT/US13/53682

§ 371 Date: Feb. 3, 2015

Related U.S. Application Data
(60) Provisional application No. 61/680,706, filed on Aug. 8, 2012.

(51) Int. Cl. G01N 30/60; G01N 33/72; G01N 33/558; G01N 33/573

(52) U.S. Cl. CPC G01N 30/6091; G01N 33/573; G01N 33/726; G01N 33/558; G01N 2333/916

(57) ABSTRACT

A compact chromatographic device and use thereof are disclosed. The device permits trapping of a first analyte while permitting passage of other analytes, followed by trapping of a second and subsequent analytes in an orderly series. Each analyte is trapped sequentially by a different chromatographic media. The process allows detection and/or measurement of each analyte without interference from other analytes previously trapped, enabling assays of analytes in a compact device without elution of analytes from the device. The compact device is pre-assembled and ready to use in a point of care or non-laboratory setting.

### FIELD OF THE INVENTION

The Field of the invention is the use of multiple chromatographic materials in sequence to achieve a compact analysis of multiple analytes.

### BACKGROUND OF THE INVENTION

U.S. Pat. No. 4,956,302 and its Reissue RE39,664 E disclose a lateral flow chromatographic binding assay device. However, the device disclosed therein does not permit individual detection and/or measurement of two or more analytes with cross-reactivity.

U.S. Pat. No. 4,960,691 discloses a chromatographic test strip for determining ligands or receptors by employing a chromatographic medium and a solvent capable of transporting reagents and/or sample. However, the chromatographic medium within a single test strip disclosed therein does not allow chromatographic separations of different analytes with cross-reactivity, and thus the single test strip therein alone cannot measure and/or detect individual analytes with cross-reactivity.

The aforementioned art do not utilize the power of traditional chromatography. They do not separate highly similar molecules within a compact device in a point of care or non-laboratory setting. There is a need for a compact device that can achieve separations of highly related multiple analytes for the point of care.

### SUMMARY OF THE INVENTION

In one aspect, the invention relates to a compact chromatographic device comprising:
a) an enclosure having a first end and a second end, comprising:
   (i) a top part having a length of Lt, a width of Wt, and a thickness of Tt;
   (ii) a bottom part having a length of Lb, a width of Wb, and a thickness of Tt, being opposite to and spaced apart from the top part at a distance of Tp;
      wherein either the top part or the bottom part has a transparent region;
   (iii) a first spacer, and
   (iv) a second spacer, being parallel to and spaced apart from the first spacer at a distance of ds, the first and the second spacers being located between the top and the bottom parts and each having a length of Lp, a width of Lp and a thickness of Tp;
b) a reservoir, located between the two spacers and near the first end of the enclosure;
c) two or more chromatographic media arranged in an orderly series, located between the spacers and the top and bottom parts of the enclosure, and located after the reservoir;
d) optionally one or more than one frit as a porous spacer, located before and/or between the chromatographic media arranged in series, and
e) a wick as a porous receiver for receiving fluid, located after the two or more chromatographic media.

The top part may further comprises a vent hole. The two or more chromatographic media may be selected from the group consisting of affinity material and ion exchange material. The device may further comprises a color-forming substrate immobilized onto at least one of the chromatographic media.

In one embodiment of the invention, at least one of the two or more chromatographic media is selected from the group consisting of a particulate substance and a porous non-particulate substance.

In another embodiment of the invention, the two or more chromatographic media and frit are in dry form.

In another embodiment of the invention, the two or more chromatographic media and/or the one or more than one frit contains an acid, a base, a salt, a buffer, an enzyme substrate, a ligand, or a protein.

In another embodiment of the invention, at least one of the two or more chromatographic media has an affinity to a glycated component. For example, the at least one of the two or more chromatographic media contains a lectin, a boronate, or an antibody.

In another embodiment of the invention, the device may comprise at least three chromatographic media, wherein the first, the second and the third chromatographic media are:
(i) a boronate, an anion exchange material, and a cation exchange material, respectively; or
(ii) lectin, boronate, and ion exchange material, respectively.

In another embodiment of the invention, the device comprises two chromatographic media, in which one is an anion exchange material and the other is a cation exchange material.

In another embodiment of the invention, the two or more chromatographic media are arranged in such an order that the first media is adapted to trap a first analyte to avoid an interference with the second analyte, the second media is adapted to trap a second analyte to avoid an interference with the third analyte, and the third media is adapted to trap the third analyte free of an interference.

In another aspect, the invention relates to a method of assaying one or more analytes in a sample, comprising:
a) diluting the sample comprising one or more analytes with a pre-measured volume of a buffer to obtain a diluted sample;
b) adding a portion of the diluted sample to the chromatographic device as aforementioned;
c) allowing the diluted sample to pass through the chromatographic media by capillarity to separate the analytes in the two or more chromatographic media;
d) optically measuring the separated analytes within the device;
e) determining the presence and/or the quantity of the separated analytes retained on the two or more chromatographic media at specific locations within the device by comparing with a standard.

In one embodiment of the invention, the method further comprises the step of allowing a color reaction to develop prior to the optically measuring step.

In another embodiment of the invention, the one or more analytes comprises an enzyme.

In another embodiment of the invention, the method comprises steps (a), (d), (e) as aforementioned, but has different steps (b) and (c) as follows:

(b) adding a portion of the diluted sample to the chromatographic device that comprises at least three chromatographic media; and

(c) allowing the diluted sample to pass through the chromatographic media by capillarity to separate the analytes in the three chromatographic media; wherein the sample is:
(i) a blood sample and the analytes are HbA1c, HbF and other variants of hemoglobin;
(ii) a blood sample from an alcoholic subject and the analytes are hemoglobin adducts of alcoholism, HbA1c, and other hemoglobin variants; or
(iii) a blood sample from a patient with galactosemia and the analytes are hemoglobin of galactosemia, HbA1c, and other hemoglobin variants.

In another embodiment of the invention, the three chromatographic media are arranged in such an order that:
(i) the first media is adapted to trap the HbA1c to avoid an interference with the HbF, the second media is adapted to trap the HbF to avoid an interference with other variants of hemoglobin, and the third media is adapted to trap the other hemoglobin variants free of an interference;
(ii) the first media is adapted to trap the hemoglobin adducts of alcoholism to avoid an interference with the HbA1c, the second media is adapted to trap the HbA1c to avoid an interference with the other hemoglobin variants, and the third media is adapted to trap the other hemoglobin variants free of an interference; or
(iii) the first media is adapted to trap the hemoglobin of galactosemia to avoid an interference with the HbA1c, the second media is adapted to trap the HbA1c to avoid an interference with the other hemoglobin variants, and the third media is adapted to trap the other hemoglobin variants free of an interference.

In another embodiment of the invention, the sample is a blood sample and the analytes are HbA1c and HbA₀, and the device has two chromatographic media arranged in such an order that the first media is adapted to trap the HbA1c to avoid an interference with the HbA₀, the second media is adapted to trap the HbA₀ free of an interference.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1A is a schematic drawing of a chromatographic device according to one embodiment of the invention.

FIG. 1B is a top view of the device shown in FIG. A.

FIG. 1C is a side view of the device shown in FIG. A.

FIG. 1D is a cross sectional view of the device shown in FIG. A.

FIG. 2A is a graph showing the results of chromatographic separation of hemoglobins on a device containing boronate and SP chromatographic media.

FIG. 2B is a graph showing the linearity of HbA1c measurements.

FIG. 3 is a bar graph showing the effect of treating the second frit with acid or base on hemoglobin migration in the SP chromatographic media.

FIG. 4 is a graph showing the results of hemoglobin separation using three chromatographic media.

FIG. 5 is a graph showing the results of separation of hemoglobins using a non-particulate chromatographic media.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term, the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

The term “chromatography” means a process of separating a mixture of analytes by passing a mobile phase containing the mixture over a stationary phase which retards the mobility of individual analytes.

The terms “chromatographic media” and “chromatographic material” are interchangeable. The chromatographic media may contain pores or do not contain pores, and may be in particulate form or non-particulate form. The chromatographic media acts as an immobilizing attractant for analytes and is made from a substrate such as, but not limited to, agarose, SEPHAROSE™, polyacrylamide, poly 2-hydroxyethyl methacrylate, polystyrene, and silica that have been synthesized or treated with an attractant. The attractant includes, but not limited to, ion exchangers (such as quarternary amines or sulfonylpropyl), lectins, and boronates. The attractant is present throughout the entire chromatographic medium uniformly, whether it is on the surface or within the pores of chromatographic medium.

The chromatographic media may be further treated with a substance that enhances specificity and efficacy in attracting an analyte of interest. For example, boronate-containing chromatographic media may be further treated with divalent cations Zn⁺² and Mg⁺².

The term “Q” means any chromatographic material with an immobilized quaternary amine as an immobilizing attractant.

The term “SP” means any chromatographic material with an immobilized sulfonylpropyl as an immobilizing attractant.

The term “use of multiple chromatographic media” means sequential use of multiple chromatographic media sequentially arranged in a single device. In some cases, the multiple chromatographic media may be identical materials but pretreated differently to allow various trapping processes in a single compact device.

The term “separation” means a chromatographic process by which one analyte is distinguished from another analyte by differential adsorption to chromatographic media.

The term “trapping” or “capturing” means immobilization of an analyte onto chromatographic media. Trapping may be a very slow movement along chromatographic media.

The term “frit” is equivalent to “porous separator material”. It is a material used to hold a chromatographic medium in place or to separate two chromatographic media. A frit may be pretreated to alter a sample solution as it moves from one chromatographic material to another.

The term “wick” means an absorbent material acting as a driving force that pulls liquid through the functional components (chromatographic material and/or frit) of the device. In the most compact form of the invention, the wick may be composed of chemically pretreated absorbent material that also functions as a chromatographic component.

The term “enclosure” means a set of walls that holds the components of the chromatographic assay in place. It has a top part, bottom part, and sides. Openings may be provided for insertion of a sample at one end of the enclosure and to permit packing of chromatographic material, frits, and the wick. Other openings may be provided in the enclosure to allow air within the enclosure to escape as sample liquid fills the device. Either the top or bottom part has a transparent portion for optical observation or measurement. The whole device, including enclosure, chromatographic material, frits, and wicks, is disposable and not to be dismantled.

The term “lateral flow” means a general analytical process whereby analytes pre-diluted in a mobile phase are permitted to pass along a generally horizontal track on which are located a variety of treatment modalities that provide some specificity to one or a series of measurements.

The term “sample” means any mixture of compounds to be analyzed and their dilutions with assay buffer. It includes, but is not limited to, biological fluids such as urine, saliva, and blood. Also included are extracts from cells, tissues, feces, foods, soil, or environmental samples (such as from ponds, lakes, rivers).

The term “analyte” means the substance to be measured in a sample. An analyte may be a discrete chemical compound or a definable extract of an organism that gives a quantifiable measurement. Analytes in a sample may be identical in structure except for a single region or chemical modification, such as Hemoglobin Ao and Hemoglobin S, which are identical except for a single beta chain Glu6 to Val amino acid change. Similarly, Hemoglobin A1c is identical to Hemoglobin Ao except that HbA1c is covalently modified by glucose. Hemoglobins Ao, S and A1c are distinct analytes that may be measured by chromatographic separation with the device of the invention.

The term “ligand” means any molecule that binds specifically to other molecules. The two molecules that bind to each other are called a ligand pair. For example, growth hormone and growth hormone receptor are two ligands in a ligand pair. In affinity chromatography, an immobilized enzyme substrate may be used as a ligand to bind its specific enzyme ligand and trap the enzyme on chromatographic media.

The term “interference” means an analyte that would be captured together with another analyte in a chromatographic medium and lead to a less specific result. Removal of an interfering analyte by a previous chromatographic medium in serial sequence enables establishment of separation specificity, which is the essence of the invention. Thus, interfering analytes can be separated and measured in the device of the invention.

The chromatographic method as used herein means complete extraction of one analyte by a chromatographic medium before passing the remaining sample on to another downstream processing chromatographic medium

A lateral flow chromatographic device of the invention uses multiple chromatographic media sequentially arranged and maintains fluid advancing integrity among chromatographic media. Each medium is designed to capture a subclass or a single analyte in a sample and may be pretreated for making analyte separation more specific. When dried, the medium retains the pretreatment at least until a sample is applied. The sequence of chromatographic media is logically arranged to allow identification of analytes by separating them in an order that eliminates interference among analytes prior to measurement.

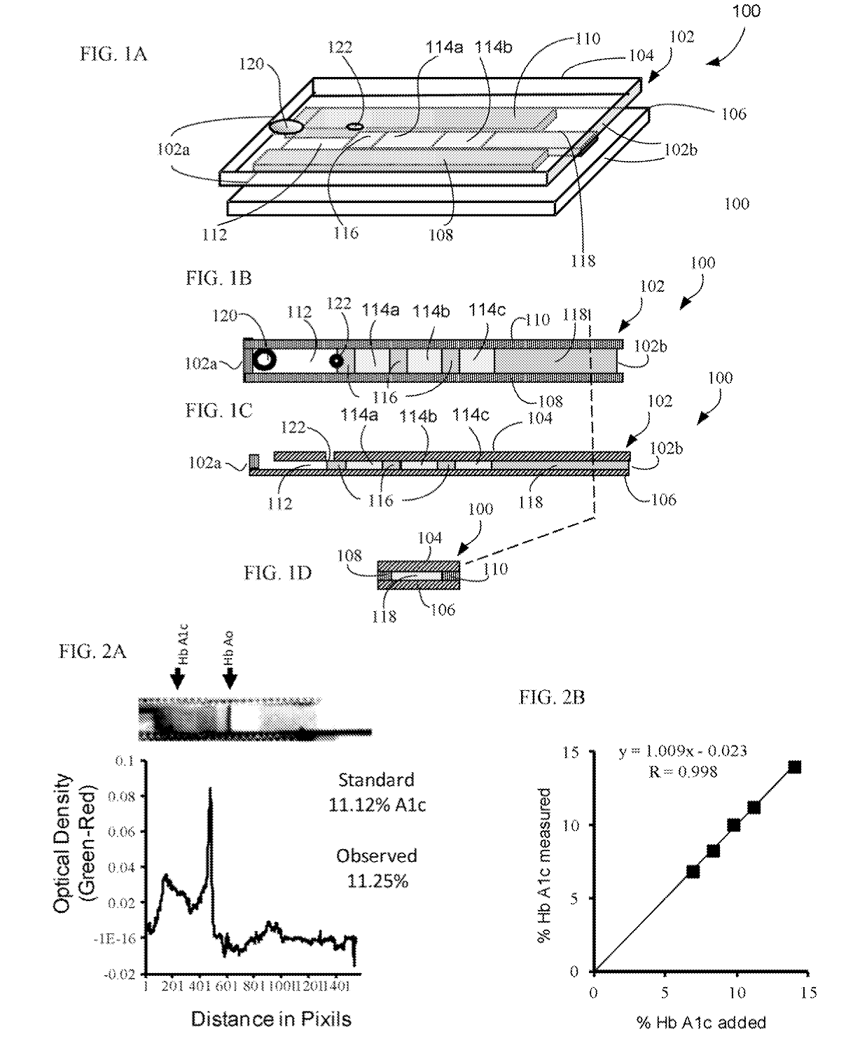

A compact chromatographic device 100 as illustrated in FIGS. 1A-D comprises: a) an enclosure 102 having a first end 102a and a second end 102b; b) a reservoir 112, located between the two spacers 108, 110 and near the first end 102a of the enclosure 102; c) two or more chromatographic media 114a, 114b, 114c arranged in an orderly series, located between the spacers 108, 110 and the top and bottom parts 104, 106 of the enclosure 102, and located after the reservoir 112; d) optional one or more than one frit 116 as a porous spacer, located before and/or between the chromatographic media arranged in series, and e) a wick 118 as a porous receiver for receiving fluid, located after the two or more chromatographic media 114a, 114b, 114c.

The two or more chromatographic media 114a, 114b, 114c, have a total length shorter than the length of the top and bottom parts 104, 106. Each chromatographic medium 114a, 114b, 114c possesses function of separating one analyte from another analyte, and such function is uniformly distributed thought the entirety of each medium 114a, 114b, 114c without interruptions. The reservoir 112 (for adding a sample), chromatographic media 114a, 114b, 114c, flit 116 are all contained within the enclosure 102.

The top part 104 and bottom part 106 may be made of a material including, but not limited to, polycarbonate, polystyrene, acetate, MYLAR™ or any suitable material. Their thickness may range from 5 to 10 mils or other suitable dimensions. Their width may range from 10 to 30 mm or any suitable range. The length may range from 70 to 90 mm or any suitable length. At least one area of the top part 104 or bottom part 106 must be transparent to allow chromatographic media to be visualized. The first spacer 108 and the second spacer 110 may be made of any material including, but not limited to, polycarbonate, polystyrene, acetate, MYLAR™, and the like. It may be formed with alternating layers of materials mentioned above and double-sided sticky tape (e.g., carpet tape) until a desired thickness is achieved. Alternatively, it may be made by having the first spacer 108 and the second spacer 110 incorporated into the top part 104 or the bottom part 106 by methods such as molding and vacuum forming. The spacer dimensions may be as follows: thickness ranging from 0.4 to 1 mm, width ranging from 5 to 10 mm, length ranging 70 to 90 mm. Other suitable dimensions may also be used. The length of the spacers 108, 110 should be no longer than the top part 104 and bottom part 106. The spacers 108, 110 may be shorter than the top part 104 and bottom part 106, and have a complex shape to accommodate the shape of the reservoir 112 and/or wick 118. The two spacers 108, 110 are adhered to, and located between, the top and the bottom parts 104,106, and are in parallel and separated from each other at a distance of 4 to 10 mm, or any suitable distance. The adhering can be achieved by an adhesive or heat sealing or any suitable method.

The top and the bottom parts 104, 106, and the two spacers 108, 110 constitute the enclosure 102. The frit 116 and wick 118 have a thickness the same as the two spacers 108, 110, and a width the same as the distance between the two spacers 108, 110. The frit 116 length may range from 2 to 5 mm, or any suitable length, and is inserted into the enclosure 102. The volume of the enclosure 102 between the first end 102a and the frit 116 or first chromatographic media 114a constitutes a reservoir 112. A hole 120 (for adding a sample) with a diameter of 1 to 3 mm, or any suitable diameter, may be cut in the top part 104 at a distance of 1 to 5 mm from the first end 102a prior to forming the enclosure 102. A second hole 122 (air vent), 0.5 to 3 mm in diameter or any convenient size, is also cut in the top part 104 above the location where the frit 116 or first media 114a is to be inserted.

The first chromatographic media 114a is packed tightly against the frit 116. The thickness and width of the packed chromatographic media 114a, 114b, 114c are determined by the thickness of and the distance between the two spacers 108, 110, respectively. The length of the chromatographic material 114 may range from 10 to 30 mm or any suitable length. Optionally, a second frit 116 with a length of 2 to 5 mm, or any suitable length, is inserted into between the first and second media 114a, 114b and pushed tightly against them. The second chromatographic medium 114b is a material different from the first chromatographic media 114a. Additional chromatographic media 114c and optional frit 116 may be similarly inserted as needed. The wick 118 is made of a porous material such as filter paper. The thickness is the same as the spacers 108, 110. The width of the wick 118 is defined by the distance between the two spacers 108, 110. The length of the wick 118 may be 5 mm or longer to cover the remaining length of the enclosure, or extending beyond.

A compact device (FIG. 1A-D) containing a series of chromatographic media 114a, 114b, 114c arranged to enhance separation specificity and to trap analytes in a logical, serial order, permits all serially trapped analytes to be measured in a small optical window without eluting the analytes from the chromatographic media.

To assay analytes, a sample is diluted in a buffer and added to the reservoir 112 located near the first end of the device 102a. Fluid movement through the frits 116, chromatographic media 114, and wick 118 is driven by capillarity. When the wick 118 is saturated with fluid, the sample uptake by the media and/or frit stops, providing a simple way of defining the amount of diluted sample used in the assay. The shape and volume of the wick 118 is therefore important in determining how much of the sample is used. For capillarity to be efficient and to perform the chromatography in a reasonable time all the materials (frit, chromatographic media and wick) need to be dry, thin and short, and preferably are limited to a thickness (or depth) of one millimeter or less, and the entire device is preferably no longer than 9 cm. The width of the chromatographic media 114 preferably is less than 1 cm because a wider cross section presents a significant risk of irregular wavy chromatographic patterns with a small amount of sample.

It is useful to separate hemoglobins in clinical samples in logical order. The determination of Hemoglobin A1c (HbA1c) as percent of total Hemoglobin is important in monitoring Diabetes Mellitus. In one embodiment of the invention, HbA1c is captured in a first chromatographic medium (a boronate) and the remaining hemoglobin is captured in a second chromatographic medium free of HbA1c.

Hemoglobin F (HbF) when present is an interference to the measurement of Hemoglobin A1c because F cannot be glycated in a usual sense. The amount of Hemoglobin F should not become part of the denominator in the determination of HbA1c %. A more specific ion exchange resin that can trap HbF is an appropriate means of determining HbF before determining the total hemoglobin. However HbA1c interferes with determination of F. The logical order of trapping is to first capture HbA1c, next to capture HbF, and finally to capture the remaining hemoglobins. Then the trapped HbA1c, HbF, remaining hemoglobins are simultaneously measured in a small optical window.

When assaying analytes which are uncolored, color-forming reagents are incorporated into chromatographic media. The color-forming reagents in the media are activated only when analytes are trapped and additional color-forming reagents are present in the sample buffer. Methods of providing a color reaction to uncolored analytes are disclosed in U.S. Pat. No. 8,318,509, which is incorporated herein by reference. In cases when the aforementioned additional color-forming reagents are incompatible with the sample or buffer, the additional color-forming reagents need to be added separately after analytes are trapped. Such additional color-forming reagents serves to improve separation of analytes and provide color formation. Table 1 illustrates how components within the enclosure of the device are orderly arranged to perform logical separations.

TABLE 1

| Order of component arrangement | | |
|---|---|---|
| within enclosure of device | Analytes | Medical use |
| Frit, Boronate, SP, wick | HbA1c, HbAo | Diabetes monitoring |
| Frit, Boronate, Q, SP, wick | HbA1c, HbF, HbAo | Diabetes monitoring with HbF |
| Frit, Boronate, carboxypaper | HbA1c, HbAo | Diabetes monitoring |
| Frit, Xylose, lectin Boronate, SP, | Alcoholic Hb, HbA1c, | Alcoholism |
| wick | HbAo | |
| Frit, Galactose lectin, Boronate, SP, | Galactose Hb, HbA1c, | Galactosemia |
| wick | HbAo | |
| Frit, Wheat germ lectin* Qt*, wick | Bone Alkaline Phosphatase, | Bone metastasis in cancer |
| | Other Alkaline Phosphatase | |
| Frit, Q*, SP*, wick | LDH1, LDH2 | Heart attack test |

*color forming system incorporated

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Drying of Microparticle Chromatographic Media

Microparticle-based chromatographic media to be dried were pretreated with aqueous solutions before dehydration. Boronate microparticles synthesized on SEPARON™ 1000 with modifications of methods described by P D G Dean, W S Johnson, and F A Middle Affinity chromatography, a practical approach IRL Press, LTD, Oxford England, 1985, Pp 35-39, and MACROPREP® Hi Q (Biorad—hereafter referred to as Q) were pretreated with distilled water. The TOYOPERL™ SP (Tosoh—hereafter referred to as SP) was washed with 10 mM HCl.

To dry the beads, they were serially washed with 50% acetone:50% distilled water, 75% acetone:25% distilled water, and 100% acetone. After decanting the acetone the residual solvent was removed by evaporation and dried microparticles were stored at 4° C. All the dried microparticles are composed of 2-Hydroxyethyl-methacrylate and ready for storage or packing into the device. In contrast, particles such as agarose gel are not readily dried by the same procedure.

### Pretreatment of Frit Porous Materials

Adsorbent paper of 0.5 mm thickness was used. Preliminary dye adsorption testing indicated that the paper contained carboxyl groups. Papers were used without or with pre-treatment with sodium metabisulfite in dilute HCl, followed by extensive washing with distilled water before drying. These papers, called “acid washed”, contain H⁺ ions covering the carboxyl groups of the papers. Some acid washed papers were further treated with 50 mM Na₂CO₃ at pH 9.5, designated as “base washed”, in which the carboxyl groups of the papers may be covered by Na⁺ rather than by H⁺ ions.

### Device Assembly.

The device was assembled as shown in FIG. 1A-D. Two clear polycarbonate pieces (75 mm by 15 mm by 5 mils thick) were used as top and bottom parts. Spacers were made from double-sided tape and MYLAR™ in alternate layers to a final thickness of 0.5 mm and cut into strips (about 75 mm long, 4 mm wide). Electrophoresis paper 0.5 mm thick (Beckman) was cut into 4 mm×50 mm strips and used as frits and wicks. Pretreatments of wicks and frits are illustrated in below examples. A first polycarbonate strip was laid down on a flat surface to serve as a bottom part of the enclosure. A first spacer was adhered onto the bottom part at one edge. A second spacer was placed onto the bottom part opposite to the first spacer in parallel at a distance of about 4 mm. A 3 to 5 mm segment of a filter paper was used as a frit, and placed between the two spacers about 20 mm from the first end of the enclosure. Two holes were cut in second sheet of polycarbonate (top part), a first hole (3 mm diameter) was made at 5 mm from the first end for adding a sample, and a second hole (2 mm diameter) made at 20 mm from the first end to serve as an air vent. The second polycarbonate piece (top part) was firmly pressed on the top of the spacers to form an enclosure, within which a space or a gap of 4 mm in width and 0.5 mm in depth is present. Dried microparticles were then added into the space within the enclosure. A second frit (0.5×4×5 mm) was inserted and used to pack the microparticles. The second type of microparticles was added following the second frit insertion. Additional layers of microparticles were added similarly as a second or more layers as desired. The wick was inserted after the last chromatographic media insertion.

### Example 1

### Two Types of Dried Microparticles Separated by a Porous Material

Devices were packed with dried boronate synthesized on SEPARON™ and dried acid washed SP cation exchange chromatographic media. Frits were in front of the boronate and before the SP. A wick was placed after the SP chromatographic media. The glycohemoglobin standard was diluted to 11.12% with HbAo and the mixture was further diluted 1:100 with 25 mM MgCl₂ 25 mM glycine buffer, pH 9.1. A 70-μl portion of the diluted standard was added to the device. No further additions were made to the device before it was scanned and digitized with a Red/Green/Blue (RGB) color printer scanner (Hewlet Packard) at a resolution of 600 DPI. The reflectance measurements were converted to optical density with a 256-increment grayscale using the program ImageJ developed at the National Institutes of Health (USA). The average optical density across the width of the packed microparticles was used for calculating the levels of hemoglobin present.

Hemoglobin was observed not to have an appreciable light absorbance in the red optical channel but does absorb light in the green and blue optical channels. The digital reflectance measurements were taken as the equivalent of transmission of light, which is reduced by hemoglobin absorbance. Optical density, which is proportional to the amount of the analyte, was calculated as the log of 100% reflectance divided by the measured reflectance at each point. Subtraction of the red optical density from the green optical density at each point was used as a correction factor to compensate for optical interferences. A separate device constructed identically was run with buffer only as a blank.

The diluted sample in the reservoir was sequentially drawn through frits, boronate and SP chromatographic media, and wick by capillarity. As the sample passed through the Boronate microparticles the glycated hemoglobin (HbA1c) is trapped. The remaining hemoglobin in the sample (HbAo) passed to the SP chromatographic medium and was trapped. In FIG. 2A, no hemoglobin remained when the diluted sample reached the wick. Quantitative analysis (FIG. 2A, bottom panel) showed two hemoglobin peaks, HbA1c in the boronate, HbAo in the SP. In between the two peaks was HbAo in passage through the boronate and frit that had not yet reached the SP when the fluid movement stopped. After accounting for the distribution of the HbA1c and HbAo, it was estimated that the sample contained 11.12% HbA1c. The experiment was repeated using a 14% HbA1c standard, a 6.9% A1c standard, and blends of the two to obtain intermediate values (FIG. 2B). The results had a linearity with the expected values with a correlation coefficient of 0.998, which indicates a successful recovery by the device of the invention for HbA1c. FIG. 2B is also a calibration curve generated by comparing the calculated concentrations of HbA1c with the known concentrations of the standards. The generated calibration curve is useful for measuring unknown samples.

### Example 2

### Pretreating Frits to Change Chromatographic Profile

Device containing boronate and SP chromatographic media were prepared as in example 1, except that the porous separators were acid or base washed before drying. As shown in FIG. 3, whether the first frit, in front of the boronate, was acid or base washed made no difference in the mobility of hemoglobin in the SP microparticles. In contrast, the second frit before the SP was important. Changing the second flit from base to acid produced a tighter binding of HbAo and decreased the mobility of the HbAo peak in the SP medium. The conclusion is that the acid second frit conditioned the sample and made the retention of hemoglobin in the SP medium stronger. Treating the second frit is equivalent to changing the buffer but without operator intervention, which makes the device of the invention compact and simple to use.

### Example 3

### More than Two Chromatographic Media for Separating Multiple Analytes in a Single Pass

The ability to measure HbA1c and hemoglobin variants without analytical interference is clinically important. The presence of HbF due to hemolytic anemia or the treatment of sickle cell anemia changes the interpretation of % HbA1c measurements. Preliminary minicolumn studies were performed to characterize which chromatographic media hemoglobin variants bind to. Three chromatographic media used were: boronate, Q anion exchange material (Biorad), and SP cation exchange material (Tosoh). HbA1c, HbF, HbA, HbS, and HbC standards, diluted in 25 mM MgCl₂, 25 mM glycine buffer, pH 9.1, each were added to minicolumns and scored for Hb retention. Table 2 shows the results, where “+” indicates hemoglobin retention and “−” denotes no retention.

TABLE 2

| | HbA1c | HbF | HbAo | HbS | HbC |
|---|---|---|---|---|---|
| Boronate | + | − | − | − | − |
| Q | +/− (~20%) | + | − | − | − |
| SP | + | + | + | + | + |

Since both HbA1c and HbF bind to Q microparticle chromatographic medium under the same conditions, the presence, absence and how much of each analyte is equivocal. Thus, further separation using more media than Q alone is needed.

Using three sequentially arranged chromatographic media solved the problem of measuring HbA1c in the presence of HbF. By arranging boronate media first, HbA1c is trapped and prevents it from interfering with HbF measurement. Having Q chromatographic medium as a second in the series allowed HbF measurement free of HbA1c interference. Arranging SP microparticles as the last in the series allowed measurement of HbAo and hemoglobin variants HbS and HbC without interference of HbF or HbA1c.

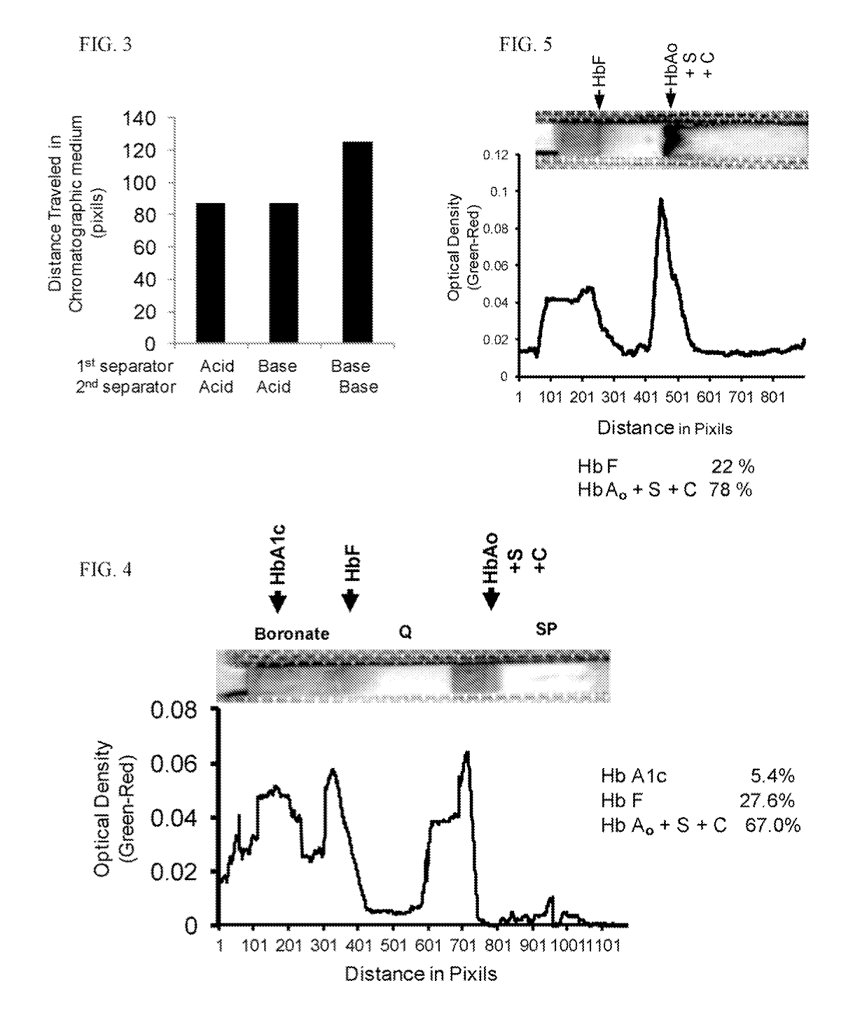

A standard containing HbA1c and HbAo (Primus, Kansas City) was mixed with a second standard containing HbF, HbAo, HbS and HbC (Primus, Kansas City), and added to the device of the invention. FIG. 4 shows three sequential peaks representing hemoglobin HbA1c, HbF and other variants, respectively. The amounts calculated from each peak agreed with the proportions expected from the mixture.

The same concept is applicable to separation of 5-deoxy-D-xylulose-5-phosphate hemoglobin adducts found in RBCs of chronic alcoholics in the presence of HbA1c and HbAo. The alcoholism hemoglobin adducts are valuable in monitoring alcoholic sobriety compliance. A lectin from the mushroom Xylaria hypoxylon that is specific for xylose (Liu et al. Biochemica et Biophysica Acta 1760:1914-1919, 2006) may be used as a first chromatographic media to trap alcoholism adducts, boronate as a second chromatographic media to trap HbA1c free of the alcoholism hemoglobin adduct interference, and SP as the last chromatographic media to trap HbAo and other hemoglobin variants. The logical arrangement of chromatographic media allows selective trapping of three analytes and their measurement in a single run. To assay a sample containing hemoglobin of galactosemia, HbA1c and HbAo analytes, the above first chromatographic media lectin is changed to mung bean seeds (Vigna radiata) lectin.

### Example 4

### Use of Non-Particulate Chromatographic Media

Electrophoresis paper (0.5 mm thick, Beckman) was precut to 4 mm×50 mm strips and soaked in 47 mM sodium meta-periodate for three hours to generate aldehydes. The presence of aldehydes was confirmed with standard Schiff reagent (Sigma). After washing with distilled water, the aldehyde containing paper was incubated for 90 minutes with 40 mM sodium hypochlorite to oxidize the aldehydes to carboxylic acids. The papers were then washed with distilled water and reduced with 13 mM sodium borohydride for 15 minutes, then washed again, treated with 10 mM HCl and dried.

Devices were made with dried Q microparticle chromatographic medium and acid-pretreated carboxylate paper (serving as medium) described above. No frit between media was used. A hemoglobin standard containing a mixture of HbF, HbAo, HbS and HbC (FASC— Primus) was diluted 1:200 and 100 μl was loaded. FIG. 5 shows HbF was retained in Q chromatographic medium and the HbAo, HbS and HbC were a band trapped in the acid-washed carboxylate paper. The HbF was calculated to be 22% of the total, close to the expected 25% value. This confirms a non-particulate chromatographic media can be used to trap all the hemoglobin variants according to the invention.

### Example 5

### Detection of Serum Isoenzymes Using Immobilized Substrates for a Color Reaction

Serum isoenzymes of alkaline phosphatase have been used as markers for the presence of a disease state such as metastatic invasion of bones and the liver. The device of this invention makes assaying isoenzymes simple.

The 2-chloro p-phenylene diamine (HRP peroxidase substrate) is immobilized on wheat germ lectin and Q chromatographic medium according to the method described in U.S. Pat. No. 8,318,509. The device is packed in the following order: a frit, the wheat germ lectin/HRP substrate chromatographic media, a second frit, the Q/HRP substrate chromatographic media and a wick. Reagents such as barium peroxide (a peroxide source) and 1-naphthyl phosphate (alkaline phosphatase substrate) are dried into separate spots in the cap of a sample dilution tube containing 2 ml of 10 mM MgCl₂, 50 mM Tris HCl, pH 8.6, 1 μg/ml HRP, 1 mg/ml bovine serum albumin buffer. The cap is then stored separately from the sample dilution tube. Ten microliters of a test serum sample is added to the buffer tube, then covered with the cap containing the dried spots of barium peroxide and 1-naphthyl phosphate, and shaken 15 seconds to mix. A 100 μl of the diluted test serum sample is added to the device and color is allowed to develop over 5 minutes. In normal samples, alkaline phosphatase with color forming activity is trapped only in the second Q/HRP media. When a patient has cancer with bone metastasis, both chromatographic media generate color.

This example illustrates the invention can assay uncolored analytes and amplifies low concentrations of analytes trapped on each type of media. The bone-specific alkaline phosphatase is the first separated out, whereas the Q/HRP media captures all other alkaline phosphatases and acts as a positive control for color development. The measurement of bone alkaline phosphatase and other alkaline phosphatases, captured by separate media, in a compact space provides information for making clinical decisions.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

1. A compact chromatographic device comprising:
a) an enclosure having a first end and a second end, comprising:
(i) a top part having a length of Lt, a width of Wt, and a thickness of Tt;
(ii) a bottom part having a length of Lb, a width of Wb, and a thickness of Tt, being opposite to and spaced apart from the top part at a distance of Tp;
wherein either the top part or the bottom part has a transparent region;
(iii) a first spacer; and
(iv) a second spacer, being parallel to and spaced apart from the first spacer at a distance of ds, the first and the second spacers being located between the top and the bottom parts and each having a length of Lp, a width of Lp and a thickness of Tp;
b) a reservoir, located between the two spacers and near the first end of the enclosure;
c) two or more chromatographic media arranged in an orderly series, located between the spacers and the top and bottom parts of the enclosure, and located after the reservoir;
d) optionally one or more than one frit as a porous spacer, located before and/or between the chromatographic media arranged in series, and
e) a wick as a porous receiver for receiving fluid, located after the two or more chromatographic media.

2. The device of claim 1, wherein the top part further comprises a vent hole.

3. The device of claim 1, wherein the two or more chromatographic media are selected from the group consisting of affinity material and ion exchange material.

4. The device of claim 1, wherein at least one of the two or more chromatographic media is selected from the group consisting of a particulate substance and a porous non-particulate substance.

5. The device of claim 1, wherein the two or more chromatographic media and frit are in dry form.

6. The device of claim 1, further comprising a color-forming substrate immobilized onto at least one of the chromatographic media.

7. The device of claim 1, wherein the two or more chromatographic media and/or the one or more than one frit contains an acid, a base, a salt, a butter, an enzyme substrate, a ligand, or a protein.

8. The device of claim 1, wherein at least one of the two or more chromatographic media has an affinity to a glycated component.

9. The device of claim 8, wherein the at least one of the two or more chromatographic media contains a lectin, a boronate, or an antibody.

10. The device of claim 1, comprising at least three chromatographic media, wherein the first, the second and the third chromatographic media are:
(i) a boronate, an anion exchange material, and a cation exchange material, respectively; or
(ii) lectin, boronate, and ion exchange material, respectively.

11. The device of claim 1, comprising two chromatographic media, in which one is an anion exchange material and the other is a cation exchange material.

12. The device of claim 1, wherein the two or more chromatographic media are arranged in such an order that the first media is adapted to trap a first analyte to avoid an interference with the second analyte, the second media is adapted to trap a second analyte to avoid an interference with the third analyte, and the third media is adapted to trap the third analyte free of an interference.

13. A method of assaying one or more analytes in a sample, comprising:
a) diluting the sample comprising one or more analytes with a pre-measured volume of a buffer to obtain a diluted sample;
b) adding a portion of the diluted sample to the chromatographic device of claim 1;
c) allowing the diluted sample to pass through the chromatographic media by capillarity to separate the analytes in the two or more chromatographic media;
d) optically measuring the separated analytes within the device;
e) determining the presence and/or the quantity of the separated analytes retained on the two or more chromatographic media at specific locations within the device by comparing with a standard.

14. A method of assaying one or more than one analyte in a sample, comprising:
a) diluting the sample comprising one or more analytes with a pre-measured volume of a buffer to obtain a diluted sample;
b) adding a portion of the diluted sample to the chromatographic device of claim 6;
c) allowing the diluted sample to pass through the chromatographic media by capillarity to separate the analytes in the two or more chromatographic media;
d) allowing a color reaction to develop;
e) optically measuring the separated analytes within the device;
f) determining the presence and/or quantity of the analytes retained on the two or more chromatographic media at specific locations within the device by comparing with a standard.

15. The method of claim 14, wherein the one or more analytes comprises an enzyme.

16. A method of assaying at least three analytes in a sample, comprising:
a) diluting the sample comprising the at least three analytes with a pre-measured volume of a buffer to obtain a diluted sample;
b) adding a portion of the diluted sample to the chromatographic device of claim 12, the device comprising at least three chromatographic media;
c) allowing the diluted sample to pass through the chromatographic media by capillarity to separate the analytes in the three chromatographic media;
d) optically measuring the separated analytes within the device;
e) determining the presence and/or quantity of the analytes retained on the three chromatographic media at specific locations within the device by comparing with a standard;
wherein the sample is:
(i) a blood sample and the analytes are HbA1c, HbF and other variants of hemoglobin;
(ii) a blood sample from an alcoholic subject and the analytes are hemoglobin adducts of alcoholism, HbA1c, and other hemoglobin variants; or
(iii) a blood sample from a patient with galactosemia and the analytes are hemoglobin of galactosemia, HbA1c, and other hemoglobin variants.

17. The method of claim 16, wherein the three chromatographic media are arranged in such an order that:
(i) the first media is adapted to trap the HbA1c to avoid an interference with the HbF, the second media is adapted to trap the HbF to avoid an interference with other variants of hemoglobin, and the third media is adapted to trap the other hemoglobin variants free of an interference;
(ii) the first media is adapted to trap the hemoglobin adducts of alcoholism to avoid an interference with the HbA1c, the second media is adapted to trap the HbA1c to avoid an interference with the other hemoglobin variants, and the third media is adapted to trap the other hemoglobin variants free of an interference; or
(iii) the first media is adapted to trap the hemoglobin of galactosemia to avoid an interference with the HbA1c, the second media is adapted to trap the HbA1c to avoid an interference with the other hemoglobin variants, and the third media is adapted to trap the other hemoglobin variants free of an interference.

18. The method of claim 13, wherein the sample is a blood sample and the analytes are HbA1c and HbA₀, and the device has two chromatographic media arranged in such an order that the first media is adapted to trap the HbA1c to avoid an interference with the HbA₀, the second media is adapted to trap the HbA₀ free of an interference.
